(19) United States
(12) Patent Application Publication
(10) Pub. No.: US 2025/0152497 A1
(43) Pub. Date: May 15, 2025

(54) SKIN MOISTURIZING FUNCTIONAL TRANSFERSOME COSMETIC COMPOSITION AND METHOD OF PREPARING THE SAME

(71) Applicant: RADKIN, SEONGNAM-SI (KR)

(72) Inventor: Meekyoung KO, Gwangju-si (KR)

(21) Appl. No.: 18/506,200

(22) Filed: Nov. 10, 2023

(51) Int. Cl. A61K 8/9789; A61K 8/34; A61K 8/35; A61K 8/44; A61K 8/49; A61K 8/65; A61K 8/68; A61K 8/73; A61K 8/92; A61K 8/99; A61Q 19/00

(52) U.S. Cl. CPC A61K 8/9789; A61K 8/347; A61K 8/35; A61K 8/44; A61K 8/442; A61K 8/498; A61K 8/65; A61K 8/68; A61K 8/735; A61K 8/922; A61K 8/99; A61Q 19/007

(57) ABSTRACT

Provided is a transfersome cosmetic composition including: a Matricaria flower extract, a Centella asiatica extract, an Arnica montana flower extract, a Scutellaria baicalensis extract, a Paeonia suffruticosa root extract, an aloe vera leaf extract, a Saccharina japonica extract, a licorice root extract, a lactobacillus lysate formed by fermenting strains belonging the lactobacillus genus, a rice bran fermentation filtrate formed by fermenting rice bran using a first composite strain including strains belonging to the bacillus genus, strains belonging to the lactobacillus genus, and yeast, a green tea fermentation extract formed by fermenting green tea using a second composite strain including strains belonging to the Aspergillus genus and strains belonging to the Lactobacillus genus, a grape seed oil, an jojoba seed oil, hydroxyacetophenone, Bifidobacterium, arginine, betaine, trehalose, sodium hyaluronate, hydrolyzed elastin, Ceramide NP, resveratrol, Genistein, tocopheryl acetate, glycerin, and purified water.

### TECHNICAL FIELD

The following disclosure relates to a skin moisturizing functional transfersome cosmetic composition and a method of preparing the same.

### BACKGROUND

Skin, which is the largest organ of the human body and occupies about 16% of the total body volume, acts as an important protective layer to protect the human body from many fatal harmful factors trying to invade the human body, for example, temperature, humidity, ultraviolet rays, and the like, while being directly in contact with an external environment. However, as we grow older, skin cells are damaged by various pollutants, strong ultraviolet rays, stress, nutritional deficiencies, and the like and cells do not proliferate properly, which causes wrinkles, loss of elasticity, keratinization, and the like on the skin.

Aging of the skin is largely classified into intrinsic aging and extrinsic aging (Cosmetics & Toiletries, 111:31-37 (1996)). Intrinsic aging is a naturally occurring aging phenomenon by gradual deterioration of the skin's physiological function with age, and is clinically characterized by decreased elasticity, rough skin texture, deep wrinkles, and pigmentation (Arch Dermatol., 130:87-95 (1994)). Extrinsic aging refers to an aging phenomenon caused by extrinsic factors such as ultraviolet rays, reactive oxygen species, and stress. Skin aging progresses rapidly by the factors, and as a result, rough winkles are produced on the skin surface and elasticity decreases rapidly.

When one dries out due to a change in temperature, a decrease in humidity, wind, or the like, the skin's physiological activity as a defensive wall against the outside decreases to promote aging. In addition, when the skin is exposed to harmful reactive oxygen species or free radicals, lipid peroxide is produced by oxidation in the body and deformation of various proteins forming the skin is caused. Meanwhile, collagen which accounts for most of the skin dermis (about 70-80% of the total dried weight of the skin) and highly elastic elastin protein are major proteins produced in fibroblast and are involved in mechanical robustness of the skin, tissue cohesion, elasticity, and the like in the dermal layer of the skin. Collagen shows a quantitative decrease with increasing age and increasing exposure to ultraviolet rays. Collagen is classified according to the shape and the structural characteristics, and the most well-known of these are types I, II, III, and IV. Collagen and elastin, which are the main structural components of the skin, have a denatured structure or a decreased biosynthesis amount by natural or external stimuli, which causes acceleration of skin aging.

In order to inhibit skin aging or slow down its progress, a cosmetic or skin protectant containing various skin protecting components such as retinol, retinoids, vitamin C, flavonoids and tocopherols, and components of medicinal herbs have been developed.

However, the components of medicinal herbs, the cosmetic, or the skin protectant are unstable themselves, and also have a big problem in stability of the cosmetic, such as precipitation and color change of the contents of a final formation and efficacy of becoming slighter due to their falling titer.

In order to solve the problems, transfersomes, which may be stably delivered without destroying physiologically active component in the pharmaceutical, cosmetic, and food fields and may contain all of the physiologically active components whether they are lipid-soluble or water-soluble, is being used. In recent years, research and development of a transmitter which may maximize a skin penetration effect are also actively progressing with the development of nanotechnology.

### RELATED ART DOCUMENTS

### Patent Documents

Patent Document 1: Korean Patent Registration No. 10-2325570

### SUMMARY

An embodiment of the present invention is directed to providing a transfersome cosmetic composition having excellent skin moisturizing functionality.

The objects of the present invention are not limited to the objects described above, and other objects that are not described above may be clearly understood by those skilled in the art from the following description.

In one general aspect, a transfersome cosmetic composition includes: a Matricaria flower extract, a Centella asiatica extract, an Arnica montana flower extract, a Scutellaria baicalensis extract, a Paeonia suffruticosa root extract, an aloe vera leaf extract, a Saccharina japonica extract, a licorice root extract, a lactobacillus lysate formed by fermenting strains belonging the lactobacillus genus, a rice bran fermentation filtrate formed by fermenting rice bran using a first composite strain including strains belonging to the bacillus genus, strains belonging to the lactobacillus genus, and yeast, a green tea fermentation extract formed by fermenting green tea using a second composite strain including strains belonging to the Aspergillus genus and strains belonging to the Lactobacillus genus, a grape seed oil, an jojoba seed oil, hydroxyacetophenone, Bifidobacterium, arginine, betaine, trehalose, sodium hyaluronate, hydrolyzed elastin, Ceramide NP, resveratrol, Genistein, tocopheryl acetate, glycerin, and purified water.

In addition, the transfersome cosmetic composition may include: 1 to 3 parts by weight of the Matricaria flower extract, 1 to 3 parts by weight of the Centella asiatica extract, 0.5 to 1.5 parts by weight of the Arnica montana flower extract, 0.5 to 1.5 parts by weight of the Scutellaria baicalensis extract, 0.5 to 1.5 parts by weight of the Paeonia suffruticosa root extract, 0.5 to 1.5 parts by weight of the aloe vera leaf extract, 0.5 to 1.5 parts by weight of the Saccharina japonica extract, 0.5 to 1.5 parts by weight of the licorice root extract, 0.5 to 1.5 parts by weight of the lactobacillus lysate formed by fermenting strains belonging the lactobacillus genus, 0.5 to 1.5 parts by weight of the rice bran fermentation filtrate formed by fermenting rice bran using a first composite strain including strains belonging to the bacillus genus, strains belonging to the lactobacillus genus, and yeast, 0.5 to 1.5 parts by weight of the green tea fermentation extract formed by fermenting green tea using a second composite strain including strains belonging to the Aspergillus genus and strains belonging to the Lactobacillus genus, 0.5 to 1.5 parts by weight of the grape seed oil, 0.5 to 1.5 parts by weight of the jojoba seed oil, 0.5 to 1.5 parts by weight of the hydroxyacetophenone, 0.5 to 1.5 parts by weight of the Bifidobacterium, 2 to 4 parts by weight of the arginine, 2 to 4 parts by weight of the betaine, 0.5 to 1.5 parts by weight of the trehalose, 4 to 6 parts by weight of the sodium hyaluronate, 3 to 5 parts by weight of the hydrolyzed elastin, 5 to 7 parts by weight of the Ceramide NP, 0.5 to 1.5 parts by weight of the resveratrol, 0.5 to 1.5 parts by weight of the Genistein, 0.5 to 1.5 parts by weight of the tocopheryl acetate, 7 to 9 parts by weight of the glycerin, and 49 to 51 parts by weight of the purified water.

In another general aspect, a method of preparing a transfersome cosmetic composition includes:
preparation of a first extract including: washing Matricaria flowers, Centella asiatica, and Arnica montana flowers, freeze-drying them at a temperature of −48 to −52° C., and crushing them to prepare a Matricaria flower powder, a Centella asiatica powder, and an Arnica montana flower powder; mixing the Matricaria flower powder, the Centella asiatica powder, and the Arnica montana flower powder at a weight ratio of 2:2:1 to prepare a first mixed powder; mixing the first mixed powder and purified water at a weight ratio of 1:9, performing stirring, and performing a first hydrothermal extraction at a temperature of 88 to 92° C. for 8 to 12 hours; freeze-drying a filtrate obtained by filtration with filter paper after performing the first hydrothermal extraction, thereby preparing an extract 1-1; mixing a solid remaining after filtration with filter paper after performing the first hydrothermal extraction with water again and performing a second hydrothermal extraction at a temperature of 118 to 122° C. for 12 to 16 hours; freeze-drying a filtrate obtained by filtration with filter paper after performing the second hydrothermal extraction, thereby preparing an extract 1-2; and mixing the extract 1-1 and the extract 1-2;
preparation of a second extract including: washing Scutellaria baicalensis, Paeonia suffruticosa roots, aloe vera leaves, Saccharina japonica, and licorice roots with boiling water and drying them at a temperature of 48 to 52° C. for 2 to 4 hours; pretreating the dried Scutellaria baicalensis, Paeonia suffruticosa roots, aloe vera leaves, Saccharina japonica, and licorice roots at a temperature of 28 to 32° C. at a pressure of 160 to 200 MPa for 3 to 7 minutes; heat-treating the pretreated Scutellaria baicalensis, Paeonia suffruticosa roots, aloe vera leaves, Saccharina japonica, and licorice roots at a temperature of 118 to 122° C. for 28 to 32 minutes; crushing the heat-treated Scutellaria baicalensis, Paeonia suffruticosa roots, aloe vera leaves, Saccharina japonica, and licorice roots to prepare a Scutellaria baicalensis powder, a Paeonia suffruticosa root powder, an aloe vera leaf powder, a Saccharina japonica powder, and a licorice root powder and mixing the Scutellaria baicalensis powder, the Paeonia suffruticosa root powder, the aloe vera leaf powder, the Saccharina japonica powder, and the licorice root powder at a weight ratio of 1:1:1:1:1 to prepare a composite powder; mixing the composite powder and purified water at a weight ratio of 1:9, performing stirring, and performing a first hydrothermal extraction at a temperature of 118 to 122° C. for 12 to 16 hours; freeze-drying a filtrate obtained by filtration with filter paper after performing the first hydrothermal extraction, thereby preparing an extract 2-1; mixing a solid remaining after the filtration with filter paper after performing the first hydrothermal extraction with water again and performing a second hydrothermal extraction at 98 to 102° C. for 8 to 12 hours; freeze-drying a filtrate obtained by filtration with filter paper after performing the second hydrothermal extraction, thereby preparing an extract 2-2; and mixing the extract 2-1 and the extract 2-2;
preparation of composite extracted water including: washing thyme leaves, morel mushrooms, and fennels and steaming them at a pressure of 0.095 to 0.105 MPa and a temperature of 240 to 260° C. for 50 to 70 minutes; heat-treating the steamed thyme leaves, morel mushrooms, and fennels at a temperature of 118 to 122° C. for 28 to 32 minutes; hot-air drying the heat-treated thyme leaves, morel mushrooms, and fennels, crushing them to prepare a thyme leaf powder, a morel mushroom powder, and a fennel powder, and mixing the thyme leaf powder, the morel mushroom powder, and the fennel powder at a weight ratio of 2:2:1 to prepare a mixed powder; mixing 13 to 17 parts by weight of the mixed powder and 83 to 87 parts by weight of the purified water to prepare a mixed liquid and aging the mixed liquid at a temperature of 38 to 42° C. for 22 to 26 hours; heat-treating the aged mixed liquid at a temperature of 88 to 92° C. for 58 to 62 minutes; and filtering the heat-treated mixed liquid to obtain a filtrate; and
mixing 4 to 6 parts by weight of the first extract, 4 to 6 parts by weight of the second extract, 0.5 to 1.5 parts by weight of a lactobacillus lysate formed by fermenting strains belonging the lactobacillus genus, 0.5 to 1.5 parts by weight of a rice bran fermentation filtrate formed by fermenting rice bran using a first composite strain including strains belonging to the bacillus genus, strains belonging to the lactobacillus genus, and yeast, 0.5 to 1.5 parts by weight of a green tea fermentation extract formed by fermenting green tea using a second composite strain including strains belonging to the Aspergillus genus and strains belonging to the Lactobacillus genus, 0.5 to 1.5 parts by weight of a grape seed oil, 0.5 to 1.5 parts by weight of an jojoba seed oil, 0.5 to 1.5 parts by weight of hydroxyacetophenone, 0.5 to 1.5 parts by weight of Bifidobacterium, 2 to 4 parts by weight of arginine, 2 to 4 parts by weight of betaine, 0.5 to 1.5 parts by weight of trehalose, 4 to 6 parts by weight of sodium hyaluronate, 3 to 5 parts by weight of hydrolyzed elastin, 5 to 7 parts by weight of Ceramide NP, 0.5 to 1.5 parts by weight of resveratrol, 0.5 to 1.5 parts by weight of Genistein, 0.5 to 1.5 parts by weight of tocopheryl acetate, 7 to 9 parts by weight of glycerin, and 49 to 51 parts by weight of the composite extracted water, and sonicating the mixture at a temperature of 43 to 47° C.

### BRIEF DESCRIPTION OF THE DRAWINGS

FIG. 1 is a photograph in which a composition of Example 3 is analyzed by fluorescence (optical) microscopy (confocal laser scanning microscopy).

FIG. 2 shows results of zeta view-analyzing the composition of Example 3 (using Electrophoresis & Brownian Motion Video Analysis Laser Scattering Microscopy).

FIG. 3 is a photograph which images a skin moisture amount using Epsilon E100 (Biox Systems Ltd., England) equipment.

### DETAILED DESCRIPTION

Hereinafter, various exemplary embodiments will be described in more detail. The exemplary embodiments described in the present specification may be variously modified. Specific exemplary embodiments may be described in detail in the detailed description. However, the specific exemplary embodiments disclosed may be only for easy understanding of various exemplary embodiments. Therefore, it should be understood that the technical idea is not limited by the disclosed specific exemplary embodiments and all equivalents or substitutes included in the spirit and technical scope of the invention are included.

Terms including ordinal numbers such as primary, secondary, first, and second may be used for describing various elements, but these elements are not limited by the terms described above. The terms described above is used only to distinguish one element from other elements.

It will be further understood that the terms “comprises” or “have” used in the specification, specify the presence of stated features, steps, operations, elements, parts, or a combination thereof, but do not preclude the presence or addition of one or more other features, numerals, steps, operations, elements, parts, or a combination thereof. It is to be understood that when one element is referred to as being “connected to” or “coupled to” another element, it may be connected directly to or coupled directly to another element or be connected to or coupled to another element having the other element intervening therebetween. On the other hand, it is to be understood that when one component is referred to as being “connected directly” to or “coupled directly” to another component, it may be connected to or coupled to another component without the other component intervening therebetween.

Besides, in the description of the disclosure, if it is determined that the detailed description of relevant known functions or components makes subject matters of the disclosure obscure, the detailed description thereof will be abbreviated and omitted.

The present invention provides a transfersome cosmetic composition including: a Matricaria flower extract, a Centella asiatica extract, an Arnica montana flower extract, a Scutellaria baicalensis extract, a Paeonia suffruticosa root extract, an aloe vera leaf extract, a Saccharina japonica extract, a licorice root extract, a lactobacillus lysate formed by fermenting strains belonging the lactobacillus genus, a rice bran fermentation filtrate formed by fermenting rice bran using a first composite strain including strains belonging to the bacillus genus, strains belonging to the lactobacillus genus, and yeast, a green tea fermentation extract formed by fermenting green tea using a second composite strain including strains belonging to the Aspergillus genus and strains belonging to the Lactobacillus genus, a grape seed oil, an jojoba seed oil, hydroxyacetophenone, Bifidobacterium, arginine, betaine, trehalose, sodium hyaluronate, hydrolyzed elastin, Ceramide NP, resveratrol, Genistein, tocopheryl acetate, glycerin, and purified water.

Hereinafter, the transfersome cosmetic composition according to the present invention will be described in detail.

It is preferred that the transfersome cosmetic composition according to the present invention includes: 1 to 3 parts by weight of the Matricaria flower extract, 1 to 3 parts by weight of the Centella asiatica extract, 0.5 to 1.5 parts by weight of the Arnica montana flower extract, 0.5 to 1.5 parts by weight of the Scutellaria baicalensis extract, 0.5 to 1.5 parts by weight of the Paeonia suffruticosa root extract, 0.5 to 1.5 parts by weight of the aloe vera leaf extract, 0.5 to 1.5 parts by weight of the Saccharina japonica extract, 0.5 to 1.5 parts by weight of the licorice root extract, 0.5 to 1.5 parts by weight of the lactobacillus lysate formed by fermenting strains belonging the lactobacillus genus, 0.5 to 1.5 parts by weight of the rice bran fermentation filtrate formed by fermenting rice bran using a first composite strain including strains belonging to the bacillus genus, strains belonging to the lactobacillus genus, and yeast, 0.5 to 1.5 parts by weight of the green tea fermentation extract formed by fermenting green tea using a second composite strain including strains belonging to the Aspergillus genus and strains belonging to the Lactobacillus genus, 0.5 to 1.5 parts by weight of the grape seed oil, 0.5 to 1.5 parts by weight of the jojoba seed oil, 0.5 to 1.5 parts by weight of the hydroxyacetophenone, 0.5 to 1.5 parts by weight of the Bifidobacterium, 2 to 4 parts by weight of the arginine, 2 to 4 parts by weight of the betaine, 0.5 to 1.5 parts by weight of the trehalose, 4 to 6 parts by weight of the sodium hyaluronate, 3 to 5 parts by weight of the hydrolyzed elastin, 5 to 7 parts by weight of the Ceramide NP, 0.5 to 1.5 parts by weight of the resveratrol, 0.5 to 1.5 parts by weight of the Genistein, 0.5 to 1.5 parts by weight of the tocopheryl acetate, 7 to 9 parts by weight of the glycerin, and 49 to 51 parts by weight of the purified water, and it is more preferred that the transfersome cosmetic composition includes: 1.5 to 2.5 parts by weight of the Matricaria flower extract, 1.5 to 2.5 parts by weight of the Centella asiatica extract, 0.8 to 1.2 parts by weight of the Arnica montana flower extract, 0.8 to 1.2 parts by weight of the Scutellaria baicalensis extract, 0.8 to 1.2 parts by weight of the Paeonia suffruticosa root extract, 0.8 to 1.2 parts by weight of the aloe vera leaf extract, 0.8 to 1.2 parts by weight of the Saccharina japonica extract, 0.8 to 1.2 parts by weight of the licorice root extract, 0.8 to 1.2 parts by weight of the lactobacillus lysate formed by fermenting strains belonging the lactobacillus genus, 0.8 to 1.2 parts by weight of the rice bran fermentation filtrate formed by fermenting rice bran using a first composite strain including strains belonging to the bacillus genus, strains belonging to the lactobacillus genus, and yeast, 0.8 to 1.2 parts by weight of the green tea fermentation extract formed by fermenting green tea using a second composite strain including strains belonging to the Aspergillus genus and strains belonging to the Lactobacillus genus, 0.8 to 1.2 parts by weight of the grape seed oil, 0.8 to 1.2 parts by weight of the jojoba seed oil, 0.8 to 1.2 parts by weight of the hydroxyacetophenone, 0.8 to 1.2 parts by weight of the Bifidobacterium, 2.5 to 3.5 parts by weight of the arginine, 2.5 to 3.5 parts by weight of the betaine, 0.8 to 1.2 parts by weight of the trehalose, 4.5 to 5.5 parts by weight of the sodium hyaluronate, 3.5 to 4.5 parts by weight of the hydrolyzed elastin, 5.5 to 6.5 parts by weight of the Ceramide NP, 0.8 to 1.2 parts by weight of the resveratrol, 0.8 to 1.2 parts by weight of the Genistein, 0.8 to 1.2 parts by weight of the tocopheryl acetate, 7.5 to 8.5 parts by weight of the glycerin, and 49.5 to 50.5 parts by weight of the purified water.

Matricaria flowers are dicotyledon plants belonging to the Campanulales order of the chrysanthemum family and is also referred to as chamomile. It is known that the plant has effects of skin soothing and suppressing evaporation of moisture in the skin when being applied to the skin.

Centella asiatica is an Apiaceae plant which is also referred to as tiger grass, and has excellent antibacterial and anti-inflammatory actions and an excellent wound healing effect due to a Madecassic acid component included in the leaves and stems of the Centella asiatica. In addition, the Centella asiatica extract strengthens a skin barrier and helps skin soothing, skin regeneration, and skin elasticity, and rapidly restores overall skin immunity and skin conditions.

Arnica montana flowers grow in the grasslands of the plateau and has a height of 20 to 30 cm. The leaves come out from the roots and spread in all directions and hang from the stem facing each other. The branches split at the end and have 1 to 3 head flowers. The flowers bloom yellow in June and July, have a diameter of 6 to 8 cm, and overall have dense pili and hairs. The flowers and the rhizomes taste bitter and include an essential oil and a resin. The flowers and the rhizomes have been used as a panacea by European folks since the old times. The plant is used in irritation of angina pectoris, in vasodilation, in relief of vasospasm, and as a hemostatic agent for bruises, hemorrhoids, and the like in Oriental medicine.

The Matricaria flower extract, the Centella asiatica extract, the Arnica montana flower extract, and the Scutellaria baicalensis extract are prepared by performing: washing the Matricaria flowers, the Centella asiatica, and the Arnica montana flowers, freeze-drying them at a temperature of −48 to −52° C., and crushing them to prepare a Matricaria flower powder, a Centella asiatica powder, and a Arnica montana flower powder; mixing the Matricaria flower powder, the Centella asiatica powder, and the Arnica montana flower powder at a weight ratio of 2:2:1 to prepare a first mixed powder; mixing the first mixed powder and purified water at a weight ratio of 1:9, performing stirring, and performing a first hydrothermal extraction at a temperature of 88 to 92° C. for 8 to 12 hours; freeze-drying a filtrate obtained by filtration with filter paper after performing the first hydrothermal extraction, thereby preparing an extract 1-1; mixing a solid remaining after the filtration with filter paper after performing the first hydrothermal extraction with water again, and performing a second hydrothermal extraction at a temperature of 118 to 122° C. for 12 to 16 hours; freeze-drying a filtrate obtained by filtration with filter paper after performing the second hydrothermal extraction, thereby preparing an extract 1-2; and mixing the extract 1-1 and the extract 1-2.

In the present invention, the Matricaria flower extract, the Centella asiatica extract, and the Arnica montana flower extract are applied to provide excellent skin moisturizing functionality.

Scutellaria baicalensis is a Lamiaceae perennial herbaceous plant, contains bioactive components such as flavonoids, wogonin, and baicalin, and has an effect of lowering heart and lung fever or stopping coughing and bleeding as a pharmacological effect. It has anti-allergenic, antioxidant, and anti-inflammatory effects on the skin and has an excellent harmful oxygen removal effect.

Paeonia suffruticosa, which is a type of seed plant belonging to the Paeonia genus, is a broadleaf shrub tree which grows about 1 cm. The leaves are double pinnate compound leaves and have a characteristic of being staggered. An important pharmacological action material of peony root bark is considered as being paeonol and its glycosides, and has a central depressant effect such as sedation, defervescence, pain relief, and antispasmodic actions and is effective in anti-inflammatory action, hemostatic action, and the like.

The main components of aloe vera leaves include anthraquinones such as aloin, aloe-emodin, aloenin, and aloesin, high molecular polysaccharides, glycoprotein, amino acids, minerals, and the like. It is known that the pharmacological effects of the aloe vera include pain relief, anti-allergy, antimicrobial action, tonic action, anti-inflammatory action, antitumor action, drop in blood sugar level, cadmium toxicity mitigation, and the like.

Saccharina japonica is known to be rich in nutrients such as iodine, vitamin B2, and glutamic acid, and it was reported that alginate which forms the cell wall of the Saccharina japonica has high viscosity, promotes intestinal peristalsis and stimulates defecation, and acts to adsorb heavy metals in the body and release them from the body.

Licorice is a perennial grass which is a dicotyledonous plant of the Rosaceae Legume family and is a medicinal plant, and the roots are reddish brown and go deep into the ground, and the stem is hairy and grows straight to about 1 m. In addition, it looks grayish white since white hair grows densely and lines and dots are scattered. The leaves are misaligned and are odd pinnate compound leaves. The number of small leaves is 7 to 17 each, and they are egg-shaped and have a pointed end. The small leaves have a length of 2 to 5 cm and a width of 1 to 3 cm, white hair, lines, and dots on both surfaces, and no teeth. The roots taste sweet and are used as sweeteners and herbal medicine.

The Scutellaria baicalensis extract, the Paeonia suffruticosa root extract, the aloe vera leaf extract, the Saccharina japonica extract, and the licorice root extract are prepared by performing: washing the Scutellaria baicalensis, the Paeonia suffruticosa roots, the aloe vera leaves, the Saccharina japonica, and the licorice roots with boiled water and drying them at a temperature of 48 to 52° C. for 2 to 4 hours; pretreating the dried Scutellaria baicalensis, Paeonia suffruticosa roots, aloe vera leaves, Saccharina japonica, and licorice roots at a temperature of 28 to 32° C. at a pressure of 160 to 200 MPa for 3 to 7 minutes; heat-treating the pretreated Scutellaria baicalensis, Paeonia suffruticosa roots, aloe vera leaves, Saccharina japonica, and licorice roots at a temperature of 118 to 122° C. for 28 to 32 minutes; crushing the heat-treated Scutellaria baicalensis, Paeonia suffruticosa roots, aloe vera leaves, Saccharina japonica, and licorice roots to prepare a Scutellaria baicalensis powder, a Paeonia suffruticosa root powder, an aloe vera leaf powder, a Saccharina japonica powder, and a licorice root powder and mixing the Scutellaria baicalensis powder, the Paeonia suffruticosa root powder, the aloe vera leaf powder, the Saccharina japonica powder, and the licorice root powder at a weight ratio of 1:1:1:1:1 to prepare a composite powder; mixing the composite powder and purified water at a weight ratio of 1:9, performing stirring, and performing a first hydrothermal extraction at a temperature of 118 to 122° C. for 12 to 16 hours; freeze-drying a filtrate obtained by filtration with filter paper after performing the first hydrothermal extraction, thereby preparing an extract 2-1; mixing a solid remaining after the filtration with filter paper after performing the first hydrothermal extraction with water again and performing a second hydrothermal extraction at 98 to 102° C. for 8 to 12 hours; freeze-drying a filtrate obtained by filtration with filter paper after performing the second hydrothermal extraction, thereby preparing an extract 2-2; and mixing the extract 2-1 and the extract 2-2.

In the present invention, the Scutellaria baicalensis extract, the Paeonia suffruticosa root extract, the aloe vera leaf extract, the Saccharina japonica extract, and the licorice root extract are applied to provide excellent skin moisturizing functionality.

In addition, it is more preferred that the cosmetic composition includes composite extraction water without the purified water.

As the composite extracted water, water extracted from thyme, morel mushrooms, and fennel is used.

Thyme (Thymus vulgaris) refers to a perennial plant in the Lamiaceae family in the Tubiflorae order of a dicotyledonous plant.

Morel mushrooms (Morchella esculenta (L) pers.) are mycorrhizal fungi which occur in bushes, gardens, roadsides, especially near fir or spruces between March and May and are distributed worldwide including Korea, Japan, and the like. The cap part of a fruiting body includes ascospores and paraphyses. The body is formed of a cap and a sack and has a height of 6 to 12 cm. The cap is light yellow and wide egg shaped, has a basket-shaped groove, and has soft flesh. The sack has a length of 4 to 5.5 cm and a width of 3 to 2.6 cm, has an almost cylindrical shape, is white or light yellowish white, and is hollow.

Fennel (Foeniculum vulgare) is a plant native to the Mediterranean coast having a height of 1.5 to 2 m, and its stem grows straight and is hollow and its leaf is long sheath-shaped with a pointed end and has a shape thinly divided into 3-4 branches like feathers. It is mainly used as food or spices and has various pharmacological effects such as treatment of female diseases, stress relief and sound sleep, and diuresis promotion.

The composite extracted water is prepared by performing: washing thyme leaves, morel mushrooms, and fennels and steaming them at a pressure of 0.095 to 0.105 MPa and a temperature of 240 to 260° C. for 50 to 70 minutes; heat-treating the steamed thyme leaves, morel mushrooms, and fennels at a temperature of 118 to 122° C. for 28 to 32 minutes; hot-air drying the heat-treated thyme leaves, morel mushrooms, and fennels, crushing them to prepare a thyme leaf powder, a morel mushroom powder, and a fennel powder, and mixing the thyme leaf powder, the morel mushroom powder, and the fennel powder at a weight ratio of 2:2:1 to prepare a mixed powder; mixing 13 to 17 parts by weight of the mixed powder and 83 to 87 parts by weight of the purified water to prepare a mixed liquid and aging the mixed liquid at a temperature of 38 to 42° C. for 22 to 26 hours; heat-treating the aged mixed liquid at a temperature of 88 to 92° C. for 58 to 62 minutes; and filtering the heat-treated mixed liquid to obtain a filtrate.

In the present invention, the composite extracted water is applied to provide excellent skin moisturizing functionality.

In addition, the present invention provides a method of preparing a transfersome cosmetic composition including:
preparation of a first extract including: washing Matricaria flowers, Centella asiatica, and Arnica montana flowers, freeze-drying them at a temperature of −48 to −52° C., and crushing them to prepare a Matricaria flower powder, a Centella asiatica powder, and an Arnica montana powder; mixing the Matricaria flower powder, the Centella asiatica powder, and the Arnica montana powder at a weight ratio of 2:2:1 to prepare a first mixed powder; mixing the first mixed powder and purified water at a weight ratio of 1:9, performing stirring, and performing a first hydrothermal extraction at a temperature of 88 to 92° C. for 8 to 12 hours; freeze-drying a filtrate obtained by filtration with filter paper after performing the first hydrothermal extraction, thereby preparing an extract 1-1; mixing a solid remaining after the filtration with filter paper after performing the first hydrothermal extraction with water again and performing a second hydrothermal extraction at a temperature of 118 to 122° C. for 12 to 16 hours; freeze-drying a filtrate obtained by filtration with filter paper after performing the second hydrothermal extraction, thereby preparing an extract 1-2; and mixing the extract 1-1 and the extract 1-2;
preparation of a second extract including: washing Scutellaria baicalensis, Paeonia suffruticosa roots, aloe vera leaves, Saccharina japonica, and licorice roots with boiling water and drying them at a temperature of 48 to 52° C. for 2 to 4 hours; pretreating the dried Scutellaria baicalensis, Paeonia suffruticosa roots, aloe vera leaves, Saccharina japonica, and licorice roots at a temperature of 28 to 32° C. at a pressure of 160 to 200 MPa for 3 to 7 minutes; heat-treating the pretreated Scutellaria baicalensis, Paeonia suffruticosa roots, aloe vera leaves, Saccharina japonica, and licorice roots at a temperature of 118 to 122° C. for 28 to 32 minutes; crushing the heat-treated Scutellaria baicalensis, Paeonia suffruticosa roots, aloe vera leaves, Saccharina japonica, and licorice roots to prepare a Scutellaria baicalensis powder, a Paeonia suffruticosa root powder, an aloe vera leaf powder, a Saccharina japonica powder, and a licorice root powder and mixing the Scutellaria baicalensis powder, the Paeonia suffruticosa root powder, the aloe vera leaf powder, the Saccharina japonica powder, and the licorice root powder at a weight ratio of 1:1:1:1:1 to prepare a composite powder; mixing the composite powder and purified water at a weight ratio of 1:9, performing stirring, and performing a first hydrothermal extraction at a temperature of 118 to 122° C. for 12 to 16 hours; freeze-drying a filtrate obtained by filtration with filter paper after performing the first hydrothermal extraction, thereby preparing an extract 2-1; mixing a solid remaining after the filtration with filter paper after performing the first hydrothermal extraction with water again and performing a second hydrothermal extraction at 98 to 102° C. for 8 to 12 hours; freeze-drying a filtrate obtained by filtration with filter paper after the second hydrothermal extraction, thereby preparing an extract 2-2; and mixing the extract 2-1 and the extract 2-2;
preparation of composite extracted water including: washing thyme leaves, morel mushrooms, and fennels and steaming them at a pressure of 0.095 to 0.105 MPa and a temperature of 240 to 260° C. for 50 to 70 minutes; heat-treating the steamed thyme leaves, morel mushrooms, and fennels at a temperature of 118 to 122° C. for 28 to 32 minutes; hot-air drying the heat-treated thyme leaves, morel mushrooms, and fennels, crushing them to prepare a thyme leaf powder, a morel mushroom powder, and a fennel powder, and mixing the thyme leaf powder, the morel mushroom powder, and the fennel powder at a weight ratio of 2:2:1 to prepare a mixed powder; mixing 13 to 17 parts by weight of the mixed powder and 83 to 87 parts by weight of the purified water to prepare a mixed liquid and aging the mixed liquid at a temperature of 38 to 42° C. for 22 to 26 hours; heat-treating the aged mixed liquid at a temperature of 88 to 92° C. for 58 to 62 minutes; and filtering the heat-treated mixed liquid to obtain a filtrate; and
mixing 4 to 6 parts by weight of the first extract, 4 to 6 parts by weight of the second extract, 0.5 to 1.5 parts by weight of a lactobacillus lysate formed by fermenting strains belonging the lactobacillus genus, 0.5 to 1.5 parts by weight of a rice bran fermentation filtrate formed by fermenting rice bran using a first composite strain including strains belonging to the bacillus genus, strains belonging to the lactobacillus genus, and yeast, 0.5 to 1.5 parts by weight of a green tea fermentation extract formed by fermenting green tea using a second composite strain including strains belonging to the Aspergillus genus and strains belonging to the Lactobacillus genus, 0.5 to 1.5 parts by weight of a grape seed oil, 0.5 to 1.5 parts by weight of an jojoba seed oil, 0.5 to 1.5 parts by weight of hydroxyacetophenone, 0.5 to 1.5 parts by weight of Bifidobacterium, 2 to 4 parts by weight of arginine, 2 to 4 parts by weight of betaine, 0.5 to 1.5 parts by weight of trehalose, 4 to 6 parts by weight of sodium hyaluronate, 3 to 5 parts by weight of hydrolyzed elastin, 5 to 7 parts by weight of Ceramide NP, 0.5 to 1.5 parts by weight of resveratrol, 0.5 to 1.5 parts by weight of Genistein, 0.5 to 1.5 parts by weight of tocopheryl acetate, 7 to 9 parts by weight of glycerin, and 49 to 51 parts by weight of the composite extracted water, and sonicating the mixture at a temperature of 43 to 47° C.

Hereinafter, the method of preparing a transfersome cosmetic composition according to the present invention will be described in detail for each step.

First, the method of preparing a transfersome cosmetic composition according to the present invention includes preparation of a first extract. The preparation of a first extract includes: washing Matricaria flowers, Centella asiatica, and Arnica montana flowers, freeze-drying them at a temperature of −48 to −52° C., and crushing them to prepare a Matricaria flower powder, a Centella asiatica powder, and an Arnica montana powder; mixing the Matricaria flower powder, the Centella asiatica powder, and the Arnica montana powder at a weight ratio of 2:2:1 to prepare a first mixed powder; mixing the first mixed powder and purified water at a weight ratio of 1:9, performing stirring, and performing a first hydrothermal extraction at a temperature of 88 to 92° C. for 8 to 12 hours; freeze-drying a filtrate obtained by filtration with filter paper after performing the first hydrothermal extraction, thereby preparing an extract 1-1; mixing a solid remaining after the filtration with filter paper after performing the first hydrothermal extraction with water again and performing a second hydrothermal extraction at a temperature of 118 to 122° C. for 12 to 16 hours; freeze-drying a filtrate obtained by filtration with filter paper after performing the second hydrothermal extraction, thereby preparing an extract 1-2; and mixing the extract 1-1 and the extract 1-2.

The first extract is prepared by a two-step hydrothermal extraction process and the effective components of the Matricaria flowers, the Centella asiatica, and the Arnica montana flowers may be easily extracted. Accordingly, it is applied to the cosmetic composition to have excellent moisturizing functionality.

Next, the method of preparing a transfersome cosmetic composition according to the present invention includes preparation of a second extract. The preparation of a second extract includes: washing Scutellaria baicalensis, Paeonia suffruticosa roots, aloe vera leaves, Saccharina japonica, and licorice roots with boiling water and drying them at a temperature of 48 to 52° C. for 2 to 4 hours; pretreating the dried Scutellaria baicalensis, Paeonia suffruticosa roots, aloe vera leaves, Saccharina japonica, and licorice roots at a temperature of 28 to 32° C. at a pressure of 160 to 200 MPa for 3 to 7 minutes; heat-treating the pretreated Scutellaria baicalensis, Paeonia suffruticosa roots, aloe vera leaves, Saccharina japonica, and licorice roots at a temperature of 118 to 122° C. for 28 to 32 minutes; crushing the heat-treated Scutellaria baicalensis, Paeonia suffruticosa roots, aloe vera leaves, Saccharina japonica, and licorice roots to prepare a Scutellaria baicalensis powder, a Paeonia suffruticosa root powder, an aloe vera leaf powder, a Saccharina japonica powder, and a licorice root powder and mixing the Scutellaria baicalensis powder, the Paeonia suffruticosa root powder, the aloe vera leaf powder, the Saccharina japonica powder, and the licorice root powder at a weight ratio of 1:1:1:1:1 to prepare a composite powder; mixing the composite powder and purified water at a weight ratio of 1:9, performing stirring, and performing a first hydrothermal extraction at a temperature of 118 to 122° C. for 12 to 16 hours; freeze-drying a filtrate obtained by filtration with filter paper after performing the first hydrothermal extraction, thereby preparing an extract 2-1; mixing a solid remaining after the filtration with filter paper after performing the first hydrothermal extraction with water again and performing a second hydrothermal extraction at 98 to 102° C. for 8 to 12 hours; freeze-drying a filtrate obtained by filtration with filter paper after the second hydrothermal extraction, thereby preparing an extract 2-2; and mixing the extract 2-1 and the extract 2-2.

The second extract is prepared by a two-step hydrothermal extraction process and the effective components of Scutellaria baicalensis, Paeonia suffruticosa roots, aloe vera leaves, Saccharina japonica, and licorice roots may be easily extracted. Accordingly, it is applied to the cosmetic composition to have excellent moisturizing functionality.

Next, the method of preparing a transfersome cosmetic composition according to the present invention includes preparation of composite extracted water. The preparation of composite extracted water includes: washing thyme leaves, morel mushrooms, and fennels and steaming them at a pressure of 0.095 to 0.105 MPa and a temperature of 240 to 260° C. for 50 to 70 minutes; heat-treating the steamed thyme leaves, morel mushrooms, and fennels at a temperature of 118 to 122° C. for 28 to 32 minutes; hot-air drying the heat-treated thyme leaves, morel mushrooms, and fennels, crushing them to prepare a thyme leaf powder, a morel mushroom powder, and a fennel powder, and mixing the thyme leaf powder, the morel mushroom powder, and the fennel powder at a weight ratio of 2:2:1 to prepare a mixed powder; mixing 13 to 17 parts by weight of the mixed powder and 83 to 87 parts by weight of the purified water to prepare a mixed liquid and aging the mixed liquid at a temperature of 38 to 42° C. for 22 to 26 hours; heat-treating the aged mixed liquid at a temperature of 88 to 92° C. for 58 to 62 minutes; and filtering the heat-treated mixed liquid to obtain a filtrate.

Next, the method of preparing a transfersome cosmetic composition according to the present invention includes: mixing 4 to 6 parts by weight of the first extract, 4 to 6 parts by weight of the second extract, 0.5 to 1.5 parts by weight of a lactobacillus lysate formed by fermenting strains belonging the lactobacillus genus, 0.5 to 1.5 parts by weight of a rice bran fermentation filtrate formed by fermenting rice bran using a first composite strain including strains belonging to the bacillus genus, strains belonging to the lactobacillus genus, and yeast, 0.5 to 1.5 parts by weight of a green tea fermentation extract formed by fermenting green tea using a second composite strain including strains belonging to the Aspergillus genus and strains belonging to the Lactobacillus genus, 0.5 to 1.5 parts by weight of a grape seed oil, 0.5 to 1.5 parts by weight of an jojoba seed oil, 0.5 to 1.5 parts by weight of hydroxyacetophenone, 0.5 to 1.5 parts by weight of Bifidobacterium, 2 to 4 parts by weight of arginine, 2 to 4 parts by weight of betaine, 0.5 to 1.5 parts by weight of trehalose, 4 to 6 parts by weight of sodium hyaluronate, 3 to 5 parts by weight of hydrolyzed elastin, 5 to 7 parts by weight of Ceramide NP, 0.5 to 1.5 parts by weight of resveratrol, 0.5 to 1.5 parts by weight of Genistein, 0.5 to 1.5 parts by weight of tocopheryl acetate, 7 to 9 parts by weight of glycerin, and 49 to 51 parts by weight of the composite extracted water, and sonicating the mixture at a temperature of 43 to 47° C.

A uniform composition including transfersomes may be formed and may be effectively absorbed into the skin by the process.

Hereinafter, the present invention will be described in more detail by the following examples.

However, the following examples are only illustrative of the content of the present invention, and the scope of the invention is not limited by the examples and the experimental examples.

### <Examples 1-4> Preparation of Cosmetic Composition

Transfersome cosmetic compositions were prepared with the compositions shown in the following Table 1, using a first extract in which a Matricaria flower extract, a Centella asiatica extract, and an Arnica montana flower extract were mixed at a weight ratio of 2:2:1, a second extract in which a Scutellaria baicalensis extract, a Paeonia suffruticosa root extract, an aloe vera leaf extract, a Saccharina japonica extract, and a licorice root extract were mixed at a weight ratio of 1:1:1:1:1, and purified water or composite extraction water.

The cosmetic composition included: 1 part by weight of a lactobacillus lysate, 1 part by weight of a rice bran fermentation filtrate, 1 part by weight of a green tea fermentation extract, 1 part by weight of a grape seed oil, 1 part by weight of a jojoba seed oil, 1 part by weight of hydroxyacetophenone, 1 part by weight of Bifidobacterium, 3 parts by weight of arginine, 3 parts by weight of betaine, 1 part by weight of trehalose, 5 parts by weight of sodium hyaluronate, 4 parts by weight of hydrolyzed elastin, 6 parts by weight of Ceramide NP, 1 part by weight of resveratrol, 1 part by weight of Genistein, 1 part by weight of tocopheryl acetate, and 8 parts by weight of glycerin.

TABLE 1

| | Example 1 | Example 2 | Example 3 | Example 4 |
|---|---|---|---|---|
| First extract | 5 | — | 5 | 5 |
| Second extract | — | 5 | 5 | 5 |
| Composite extraction | — | — | — | 50 |
| water | | | | |
| Purified water | 55 | 55 | 50 | — |

### <Experimental Example 1> Analysis of Transfersome

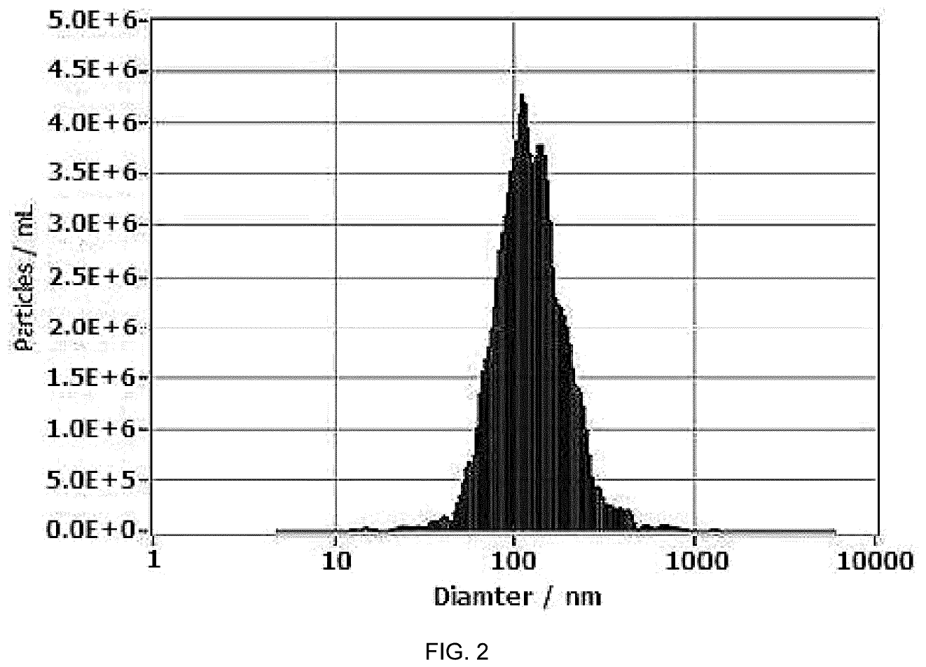

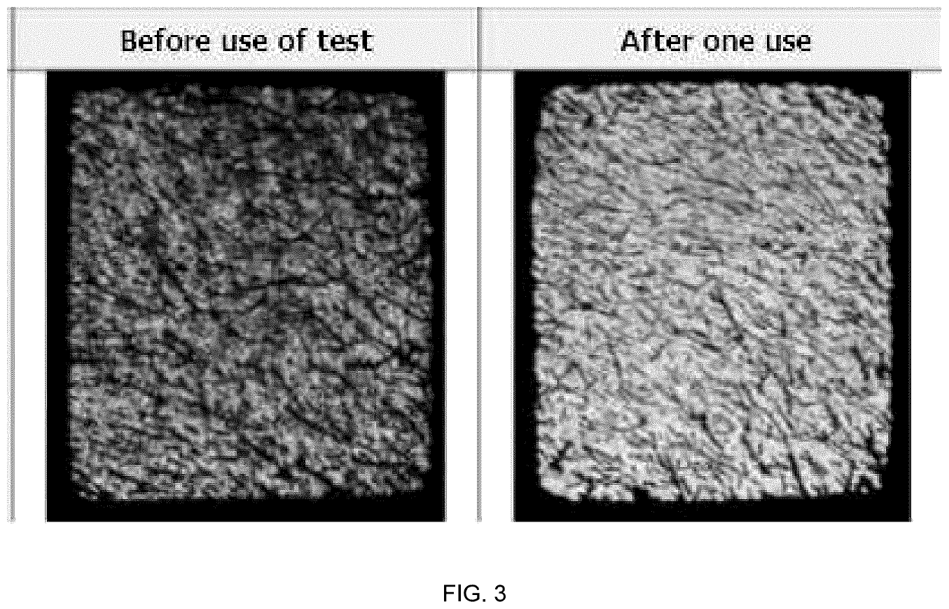

In order to confirm the shape of the transfersome cosmetic composition prepared in Example 3, fluorescence (optical) microscopy (confocal laser scanning microscopy) analysis and zeta view analysis (using Electrophoresis & Brownian Motion Video Analysis Laser Scattering Microscopy) were performed, and the results are shown in FIGS. 2 and 3.

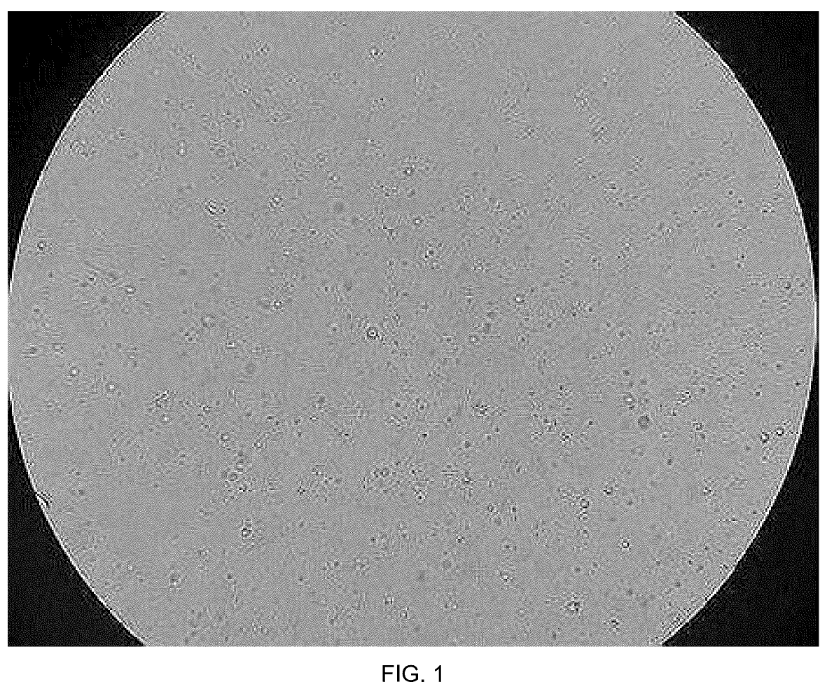

As shown in FIGS. 1 and 2, it was confirmed that the transfersome cosmetic composition according to the present invention had a particle size of 111.8 to 139.8 nm.

### <Experimental Example 2> Evaluation of Skin Moisturizing Improvement Efficacy

In order to analyze the skin moisturizing improvement efficacy of the cosmetic compositions prepared in Examples 1 to 4, the experiment was performed using an Epsilon E100 (Biox Systems Ltd., England) device.

Epsilon E100 (Biox Systems Ltd., England) is equipment which images and expresses a skin moisture amount using a CMOS fingerprint identification sensor having a 50 μm special resolution. The capacitance measurement principle was used, and a skin surface moisture amount in contact with a sensor in an area of 12.8 mm×15 mm was calculated as an average permittivity (E) value.

Specifically, tested areas of (cheek area) 96 adult women (24 people each using 4 samples) were washed and cleaned with lukewarm water, were allowed to stand for 30 minutes or more under the constant temperature and humidity (22±2° C., 50±5%) conditions to sufficiently stabilize their skin, and were tested, the average permittivity (ε) of one cheek area was measured before using the cosmetic compositions prepared in Examples 1 to 4 and after using the compositions once, and the results are shown in FIG. 3 and the following Table 2. FIG. 3 is the results of using the cosmetic composition prepared in Example 3.

TABLE 2

| | Average permittivity (ε) | | | |
|---|---|---|---|---|
| | Example 1 | Example 2 | Example 3 | Example 4 |
| Before use | 21.42 | 21.40 | 21.24 | 21.82 |
| After one use | 35.56 | 35.78 | 38.23 | 40.68 |

As shown in FIG. 3 and Table 2, it was confirmed that the cosmetic composition according to the present invention has a skin moisturizing improvement effect.

### <Experimental Example 3> Evaluation of Deep Skin Moisturizing Improvement Efficacy

In order to analyze the skin moisturizing improvement efficacy of the cosmetic compositions prepared in Examples 1 to 4, the experiment was performed using a Moisturemeter D (Delfin Technologies Ltd., Finland)) device.

Moisturemeter D (Delfin Technologies Ltd., Finland) is an instrument which is formed of an electronic control device and a probe which measures the dielectric constant of the skin tissue (tissue dielectric constant, TDC), and delivers high-frequency electromagnetic waves of 265 MHz to the skin through the probe and evaluates the amount of moisture in the skin through the reflected electromagnetic waves. The measurement depth may be adjusted depending on the size of the probe, and the resulting value is measured to be higher with the increased moisture content.

Specifically, tested areas (cheek area) of 96 adult women (24 people each using 4 samples) were washed and cleaned with lukewarm water, were allowed to stand for 30 minutes or more under the constant temperature and humidity (22±2° C., 50±5%) conditions to sufficiently stabilize their skin, and were tested, the amount of moisture in the skin (TDC) of one cheek area was measured before using the cosmetic compositions prepared in Examples 1 to 4 and after using the compositions once, and the results are shown in the following Table 3.

TABLE 3

| | Deep skin moisture content (TDC) | | | |
|---|---|---|---|---|
| | Example 1 | Example 2 | Example 3 | Example 4 |
| Before use | 34.72 | 34.20 | 33.98 | 34.58 |
| After one use | 36.76 | 36.68 | 37.50 | 39.88 |

As shown in Table 3, it was confirmed that the cosmetic composition according to the present invention had a deep skin moisturizing improvement effect.

### <Experimental Example 4> Evaluation of Skin Adverse Reaction

Whether adverse skin reactions occurred during the experiments of Experimental Examples 2 and 3 and the use of drugs in combination which may affect the test were confirmed. When the adverse reaction occurred, the tester immediately informed a person in charge of the test, and the person in charge took appropriate action after confirmation and then determined whether the tester continued participation in the test.

During the period of the tests of Experimental Examples 2 and 3, after using the cosmetic compositions of Examples 1 to 4 on the test subject, an adverse reaction such as allergic contact dermatitis or irritant contact dermatitis was not reported or observed.

The transfersome cosmetic composition according to the present invention has excellent skin moisturizing functionality.

What is claimed is:

1. A transfersome cosmetic composition comprising: a Matricaria flower extract, a Centella asiatica extract, an Arnica montana flower extract, a Scutellaria baicalensis extract, a Paeonia suffruticosa root extract, an aloe vera leaf extract, a Saccharina japonica extract, a licorice root extract, a lactobacillus lysate formed by fermenting strains belonging the lactobacillus genus, a rice bran fermentation filtrate formed by fermenting rice bran using a first composite strain including strains belonging to the bacillus genus, strains belonging to the lactobacillus genus, and yeast, a green tea fermentation extract formed by fermenting green tea using a second composite strain including strains belonging to the Aspergillus genus and strains belonging to the Lactobacillus genus, a grape seed oil, an jojoba seed oil, hydroxyacetophenone, Bifidobacterium, arginine, betaine, trehalose, sodium hyaluronate, hydrolyzed elastin, Ceramide NP, resveratrol, Genistein, tocopheryl acetate, glycerin, and purified water.

2. The transfersome cosmetic composition of claim 1, wherein the transfersome cosmetic composition includes: 1 to 3 parts by weight of the Matricaria flower extract, 1 to 3 parts by weight of the Centella asiatica extract, 0.5 to 1.5 parts by weight of the Arnica montana flower extract, 0.5 to 1.5 parts by weight of the Scutellaria baicalensis extract, 0.5 to 1.5 parts by weight of the Paeonia suffruticosa root extract, 0.5 to 1.5 parts by weight of the aloe vera leaf extract, 0.5 to 1.5 parts by weight of the Saccharina japonica extract, 0.5 to 1.5 parts by weight of the licorice root extract, 0.5 to 1.5 parts by weight of the lactobacillus lysate formed by fermenting strains belonging the lactobacillus genus, 0.5 to 1.5 parts by weight of the rice bran fermentation filtrate formed by fermenting rice bran using a first composite strain including strains belonging to the bacillus genus, strains belonging to the lactobacillus genus, and yeast, 0.5 to 1.5 parts by weight of the green tea fermentation extract formed by fermenting green tea using a second composite strain including strains belonging to the Aspergillus genus and strains belonging to the Lactobacillus genus, 0.5 to 1.5 parts by weight of the grape seed oil, 0.5 to 1.5 parts by weight of the jojoba seed oil, 0.5 to 1.5 parts by weight of the hydroxyacetophenone, 0.5 to 1.5 parts by weight of the Bifidobacterium, 2 to 4 parts by weight of the arginine, 2 to 4 parts by weight of the betaine, 0.5 to 1.5 parts by weight of the trehalose, 4 to 6 parts by weight of the sodium hyaluronate, 3 to 5 parts by weight of the hydrolyzed elastin, 5 to 7 parts by weight of the Ceramide NP, 0.5 to 1.5 parts by weight of the resveratrol, 0.5 to 1.5 parts by weight of the Genistein, 0.5 to 1.5 parts by weight of the tocopheryl acetate, 7 to 9 parts by weight of the glycerin, and 49 to 51 parts by weight of the purified water.

3. A method of preparing a transfersome cosmetic composition, the method comprising:
preparation of a first extract including: washing Matricaria flowers, Centella asiatica, and Arnica montana flowers, freeze-drying them at a temperature of −48 to −52° C., and crushing them to prepare a Matricaria flower powder, a Centella asiatica powder, and an Arnica montana flower powder; mixing the Matricaria flower powder, the Centella asiatica powder, and the Arnica montana flower powder at a weight ratio of 2:2:1 to prepare a first mixed powder; mixing the first mixed powder and purified water at a weight ratio of 1:9, performing stirring, and performing a first hydrothermal extraction at a temperature of 88 to 92° C. for 8 to 12 hours; freeze-drying a filtrate obtained by filtration with filter paper after performing the first hydrothermal extraction, thereby preparing an extract 1-1; mixing a solid remaining after the filtration with filter paper after performing the first hydrothermal extraction with water again and performing a second hydrothermal extraction at a temperature of 118 to 122° C. for 12 to 16 hours; freeze-drying a filtrate obtained by filtration with filter paper after performing the second hydrothermal extraction, thereby preparing an extract 1-2; and mixing the extract 1-1 and the extract 1-2;
preparation of a second extract including: washing Scutellaria baicalensis, Paeonia suffruticosa roots, aloe vera leaves, Saccharina japonica, and licorice roots with boiling water and drying them at a temperature of 48 to 52° C. for 2 to 4 hours; pretreating the dried Scutellaria baicalensis, Paeonia suffruticosa roots, aloe vera leaves, Saccharina japonica, and licorice roots at a temperature of 28 to 32° C. at a pressure of 160 to 200 MPa for 3 to 7 minutes; heat-treating the pretreated Scutellaria baicalensis, Paeonia suffruticosa roots, aloe vera leaves, Saccharina japonica, and licorice roots at a temperature of 118 to 122° C. for 28 to 32 minutes; crushing the heat-treated Scutellaria baicalensis, Paeonia suffruticosa roots, aloe vera leaves, Saccharina japonica, and licorice roots to prepare a Scutellaria baicalensis powder, a Paeonia suffruticosa root powder, an aloe vera leaf powder, a Saccharina japonica powder, and a licorice root powder and mixing the Scutellaria baicalensis powder, the Paeonia suffruticosa root powder, the aloe vera leaf powder, the Saccharina japonica powder, and the licorice root powder at a weight ratio of 1:1:1:1:1 to prepare a composite powder; mixing the composite powder and purified water at a weight ratio of 1:9, performing stirring, and performing a first hydrothermal extraction at a temperature of 118 to 122° C. for 12 to 16 hours; freeze-drying a filtrate obtained by filtration with filter paper after performing the first hydrothermal extraction, thereby preparing an extract 2-1; mixing a solid remaining after the filtration with filter paper after performing the first hydrothermal extraction with water again and performing a second hydrothermal extraction at 98 to 102° C. for 8 to 12 hours; freeze-drying a filtrate obtained by filtration with filter paper after performing the second hydrothermal extraction, thereby preparing an extract 2-2; and mixing the extract 2-1 and the extract 2-2;
preparation of composite extracted water including: washing thyme leaves, morel mushrooms, and fennels and steaming them at a pressure of 0.095 to 0.105 MPa and a temperature of 240 to 260° C. for 50 to 70 minutes; heat-treating the steamed thyme leaves, morel mushrooms, and fennels at a temperature of 118 to 122° C. for 28 to 32 minutes; hot-air drying the heat-treated thyme leaves, morel mushrooms, and fennels, crushing them to prepare a thyme leaf powder, a morel mushroom powder, and a fennel powder, and mixing the thyme leaf powder, the morel mushroom powder, and the fennel powder at a weight ratio of 2:2:1 to prepare a mixed powder; mixing 13 to 17 parts by weight of the mixed powder and 83 to 87 parts by weight of the purified water to prepare a mixed liquid and aging the mixed liquid at a temperature of 38 to 42° C. for 22 to 26 hours; heat-treating the aged mixed liquid at a temperature of 88 to 92° C. for 58 to 62 minutes; and filtering the heat-treated mixed liquid to obtain a filtrate; and
mixing 4 to 6 parts by weight of the first extract, 4 to 6 parts by weight of the second extract, 0.5 to 1.5 parts by weight of a lactobacillus lysate formed by fermenting strains belonging the lactobacillus genus, 0.5 to 1.5 parts by weight of a rice bran fermentation filtrate formed by fermenting rice bran using a first composite strain including strains belonging to the bacillus genus, strains belonging to the lactobacillus genus, and yeast, 0.5 to 1.5 parts by weight of a green tea fermentation extract formed by fermenting green tea using a second composite strain including strains belonging to the Aspergillus genus and strains belonging to the Lactobacillus genus, 0.5 to 1.5 parts by weight of a grape seed oil, 0.5 to 1.5 parts by weight of an jojoba seed oil, 0.5 to 1.5 parts by weight of hydroxyacetophenone, 0.5 to 1.5 parts by weight of Bifidobacterium, 2 to 4 parts by weight of arginine, 2 to 4 parts by weight of betaine, 0.5 to 1.5 parts by weight of trehalose, 4 to 6 parts by weight of sodium hyaluronate, 3 to 5 parts by weight of hydrolyzed elastin, 5 to 7 parts by weight of Ceramide NP, 0.5 to 1.5 parts by weight of resveratrol, 0.5 to 1.5 parts by weight of Genistein, 0.5 to 1.5 parts by weight of tocopheryl acetate, 7 to 9 parts by weight of glycerin, and 49 to 51 parts by weight of the composite extracted water, and sonicating the mixture at a temperature of 43 to 47° C.
